(19) United States
(12) Patent Application Publication
(10) Pub. No.: US 2024/0342068 A1
(43) Pub. Date: Oct. 17, 2024

(54) CYCLODEXTRIN-BASED ETHYL LAUROYL ARGINATE (LAE) CLATHRATE, AND PREPARATION METHOD AND USE THEREOF

(71) Applicant: NANJING UNIVERSITY OF FINANCE & ECONOMICS, Nanjing (CN)

(72) Inventors: Xiaozhi TANG, Nanjing (CN); Yaoyao XU, Nanjing (CN); Yifu CHU, Nanjing (CN); Kehong HOU, Nanjing (CN); Linghan MENG, Nanjing (CN); Chengcheng GAO, Nanjing (CN); Jianmin ZHOU, Nanjing (CN)

(21) Appl. No.: 18/037,198

(22) PCT Filed: Jun. 9, 2022
(86) PCT No.: PCT/CN2022/097863

§ 371 Date: May 16, 2023

(30) Foreign Application Priority Data
Mar. 31, 2022 (CN) 202210335067.5

(51) Int. Cl. A61K 8/44; A23L 3/3526; A23L 29/30; A61K 8/73; A61Q 19/00

(52) U.S. Cl. CPC A61K 8/442; A23L 3/3526; A23L 29/35; A61K 8/738; A61Q 19/00; A61K 2800/10; A61K 2800/524; A61K 2800/56

(57) ABSTRACT

A cyclodextrin-based ethyl lauroyl arginate (LAE) clathrate, and a preparation method and use thereof, belonging to the technical field of food and cosmetic additives. In the clathrate, a cyclodextrin compound is clathrated on the outside of an LAE compound to form a steric hindrance effect. In this way, the interaction between head-end cations of the LAE compound and anion substances is reduced, thereby significantly improving an antibacterial performance of the LAE compound. Moreover, the clathrate has a special structure in which a head is hydrophilic, a tail is hydrophobic, an outer wall of a cavity of the cyclodextrin compound is hydrophilic, and an inner wall of the cavity of the cyclodextrin compound is hydrophobic. This significantly improves an emulsification performance and a low-temperature solubility of the LAE compound, and reduces a self-aggregation effect of the LAE compound. Therefore, the clathrate has excellent thermal stability, acid-base stability, and low-temperature storage stability, and shows desirable application prospects in food and cosmetics.

### CROSS REFERENCE TO RELATED APPLICATION

The present application claims priority to Chinese Patent Application No. CN202210335067.5 filed to the China National Intellectual Property Administration (CNIPA) on Mar. 31, 2022 and entitled “CYCLODEXTRIN-BASED ETHYL LAUROYL ARGINATE (LAE) CLATHRATE, AND PREPARATION METHOD AND USE THEREOF”, which is incorporated herein by reference in its entirety.

### TECHNICAL FIELD

The present disclosure relates to the technical field of food and cosmetic additives, in particular to a cyclodextrin-based ethyl lauroyl arginate (LAE) clathrate, and a preparation method and use thereof.

### BACKGROUND

Ethyl lauroyl arginate (LAE) is a compound obtained by enzymatic catalysis or chemical synthesis of lauric acid, L-arginine, and ethanol. The LAE has a broad-spectrum antibacterial activity due to an ability to damage the cell membranes of microorganisms by cations. The LAE can form LAE salt derivatives with hydrochloric acid, lactic acid, citric acid, ascorbic acid, and fatty acids, and the derivatives have longer-lasting antibacterial properties than those of the LAE.

For example, ethyl lauroyl arginate hydrochloride (LAE·HCl) is a white hygroscopic solid with a solubility of less than 2% in water at room temperature and a melting point of 42° C. to 45° C. LAE·HCl can maintain desirable chemical stability at a pH value of 3 to 7. LAE·HCl has an amphiphilic structure in which head cations are hydrophilic and a tail carbon chain is hydrophobic, and shows a certain foaming and decontamination abilities. Therefore, the LAE·HCl is approved as a cosmetic surfactant. In addition, LAE·HCl is decomposed into fatty acids, ethanol, and amino acids in humans, animals, and natural environments. As a result, the LAE·HCl is an environmental-friendly substance that can be added to food as an antibacterial preservative. However, due to the cationic nature, LAE·HCl easily interacts with anionic components in the food, resulting in a significant decrease in antibacterial ability. The LAE·HCl has a solubility decrease at a pH value outside the range of 3 to 7, high ionic strength, and low temperature, and is easy to crystallize out from the solution, thus affecting the antibacterial properties of the LAE·HCl. Furthermore, despite having a certain emulsifying properties, the LAE·HCl shows poor emulsifying ability. Meanwhile, at a higher working concentration, the LAE·HCl has a bitter taste and affects the quality of food. These defects limit the scope of practical application of the LAE·HCl in the field of food and cosmetics.

At present, there are few researches on LAE at home and abroad. Most of the researches are based on traditional treatments of the LAE with acid, alkali, salt, or esterification groups, and can only improve some of the performances of LAE to a certain extent. However, the antibacterial and emulsifying properties of the LAE are still not satisfactory enough to completely solve the above defects. Moreover, the preparation method has higher cost and complicated process. For example, Chinese patent CN201810648982.3 disclosed a preparation method of an LAE ion pair compound derivative by conducting a reaction on the LAE with an organic acid. The derivative is used as an antibacterial agent for poultry and aquatic products. Chinese patent CN201610920777.9 combined the LAE·HCl and glycolic acid to prepare LAE glycolate. The product can be used as an antibacterial agent as well as a moisturizer. However, the prior art improves antibacterial properties by acidifying the LAE, but does not solve the problem that LAE·HCl is easy to interact with anionic components in food, resulting in decreased antibacterial properties. Moreover, the prior art also does not avoid the low emulsification ability of LAE·HCl. In Chinese patent CN201510493630.1, the LAE·HCl was used as a core material, and sodium starch octenyl succinate (SSOS) and cyclodextrin were used as a wall material, which were mixed with an anticaking agent, a dispersant, and an antioxidant and then spray-dried to obtain a microcapsule powder. This technology improves water solubility of the product at low temperature, but does not have research on the improvement of microcapsule structure and antibacterial properties. Asker et al (Asker D., Weiss J., Mclements D. J. Formation and stabilization of antimicrobial delivery systems based on electrostatic complexes of cationic-non-ionic mixed micelles and anionic polysaccharides [J]. J Agric Food Chem, 2011, 59 (3):1041-1049.) reported use of the LAE·HCl and pectin in forming an electrostatic complex. Although the electrostatic complex avoids the interaction between the LAE·HCl and other components to a certain extent, the antibacterial properties of the LAE·HCl may also be affected.

Therefore, it is of great significance to develop an LAE derivative with desirable antibacterial and emulsifying properties.

### SUMMARY

In view of this, an objective of the present disclosure is to provide a cyclodextrin-based LAE clathrate, and a preparation method and use thereof. In the present disclosure, the cyclodextrin-based LAE clathrate has excellent antibacterial and emulsifying properties.

To achieve the above objective, the present disclosure provides the following technical solutions:

The present disclosure provides a cyclodextrin-based LAE clathrate, including a cyclodextrin compound and an LAE compound penetrating a cavity of the cyclodextrin compound.

Preferably, the LAE compound is selected from the group consisting of LAE and an LAE salt.

Preferably, the LAE salt is selected from the group consisting of LAE hydrochloride, LAE lactate. LAE citrate, LAE ascorbate, and an LAE fatty acid salt.

Preferably, the cyclodextrin compound is selected from the group consisting of α-cyclodextrin, β-cyclodextrin, γ-cyclodextrin, hydroxypropyl-cyclodextrin, methyl-cyclodextrin, and glucosyl-cyclodextrin.

The present disclosure further provides a preparation method of the cyclodextrin-based LAE clathrate, including the following steps:

mixing the cyclodextrin compound, the LAE compound, and water, and conducting clathration to obtain the cyclodextrin-based LAE clathrate.

Preferably, the cyclodextrin compound and the LAE compound are at a molar ratio of (0.5-6):1.

Preferably, the mixing specifically includes: dissolving the cyclodextrin compound in the water to obtain a cyclodextrin compound solution; and dissolving the LAE compound in the cyclodextrin compound solution.

Preferably, the cyclodextrin compound solution has a mass concentration of 1% to 10%.

Preferably, the clathration is conducted at 25° C. to 80° C. for 5 min to 24 h.

Preferably, the clathration is conducted under stirring, high-speed dispersion, ultrasonic treatment, or high-pressure micro-jet;
the stirring is conducted at 300 rpm to 900 rpm for 5 h to 24 h;
the high-speed dispersion is conducted at 10,000 rpm to 18,000 rpm for 5 min to 20 min;
the ultrasonic treatment is conducted at 200 W to 750 W for 5 min to 10 min; and

the high-pressure micro-jet is conducted at 60 MPa to 100 MPa for 3 to 7 cycles.

Preferably, the preparation method further includes the following step after the clathration is completed: drying a reaction solution obtained from the clathration to obtain the cyclodextrin-based LAE clathrate.

Preferably, the drying includes freeze-drying, spray-drying, or vacuum-drying;
the freeze-drying is conducted at −80° C. to −60° C.;
the spray-drying is conducted in an atomizer at a pressure of 0.7 MPa to 1.25 MPa; the atomizer has an inlet temperature of 140° C. to 180° C., an outlet temperature of 90° C. to 110° C., and a rotational frequency of 20 Hz to 40 Hz; a feeding speed of the reaction solution is controlled by a rotating speed of a feed pump, and the rotating speed of the feed pump is 15 rpm to 30 rpm; and
the vacuum-drying is conducted under a vacuum degree of 0.05 MPa to 0.09 MPa at 50° C. to 90° C.

The present disclosure further provides use of the cyclodextrin-based LAE clathrate or a cyclodextrin-based LAE clathrate prepared by the preparation method as an additive in food or cosmetics.

The present disclosure provides a cyclodextrin-based LAE clathrate, including a cyclodextrin compound and an LAE compound penetrating a cavity of the cyclodextrin compound. In the present disclosure, in the cyclodextrin compound, the hydroxyl groups at positions C-2, C-3, and C-6 all face the outside of a cylinder cavity wall, showing hydrophilic properties of a cavity outer wall. The hydrogen atoms (H-3 and H-5) are located on an inner side of the cylinder cavity wall, showing hydrophobic properties of a cavity inner wall. The entire cyclodextrin molecule presents a hollow cylindrical structure with a wide opening at an upper end and a narrow opening at a lower end, as well as a hydrophilic outside and a hydrophobic inside. Precisely, due to this unique cavity structure with the hydrophilic outside and the hydrophobic inside, the cyclodextrin compound can form the cyclodextrin-based LAE clathrate with the LAE compound having an amphiphilic structure with a cationic hydrophilic head and a long-chain hydrophobic tail. The cyclodextrin compound clathrated outside a middle section of the LAE compound has a steric hindrance effect, which can reduce the interaction between head-end hydrophilic cations of the LAE compound and the anion components. As a result, the antibacterial properties of the LAE compound are significantly improved. Moreover, the clathrate has a special structure in which a head is hydrophilic, a tail is hydrophobic, an outer wall of a cavity of the cyclodextrin compound is hydrophilic, and an inner wall of the cavity of the cyclodextrin compound is hydrophobic. Therefore, the clathrate has excellent emulsification performance, low-temperature solubility, thermal stability, acid-base stability, and low temperature storage stability. In this way, a self-aggregation effect of the LAE compound in an aqueous solution is reduced, and a dispersion degree of the LAE compound is improved, thereby expanding an application range of the LAE compound in food and cosmetics. This also helps to mask the taste and control a release of the LAE compound.

The present disclosure further provides a preparation method of the cyclodextrin-based LAE clathrate. In the present disclosure, the preparation method has simple operations, wide sources of preparation raw materials, and a low cost. The preparation method uses water as a solvent, which is environmental-friendly and suitable for industrial production.

### BRIEF DESCRIPTION OF THE DRAWINGS

FIG. 1 shows a structural schematic diagram of the cyclodextrin-based LAE clathrate;

FIG. 2 shows Fourier transform infrared spectrograms (A) and X-ray diffraction (XRD) patterns (B) of raw materials (HPβCD and LAE) and prepared products in Example 1 and Comparative Example 1; where a is the HPβCD, b is the LAE, c is a physical mixture of the HPβCD/LAE, and d is an HPβCD/LAE clathrate;

FIG. 3 shows ¹H NMR spectrograms of the raw materials and the HPβCD/LAE clathrate in Example 1;

FIG. 4 shows an antibacterial activity of the HPβCD/LAE clathrate prepared in Example 1 to Staphylococcus aureus when xanthan gum is used as an interfering substance;

FIG. 5 shows a comparison result of emulsifying properties of the HPβCD/LAE clathrate prepared in Example 1 with other emulsifiers; and

FIG. 6 shows a turbidity (A), a particle size (B), and a low-temperature storage appearance (C) of the raw material LAE and the HPβCD LAE clathrate in each of the examples at a pH value of 1 to 11.

### DETAILED DESCRIPTION OF THE EMBODIMENTS

The present disclosure provides a cyclodextrin-based LAE clathrate, including a cyclodextrin compound and an LAE compound penetrating a cavity of the cyclodextrin compound.

In the present disclosure, the LAE compound is preferably selected from the group consisting of LAE and an LAE salt. The LAE salt is preferably selected from the group consisting of LAE hydrochloride, LAE lactate, LAE citrate, LAE ascorbate, and an LAE fatty acid salt.

In the present disclosure, the cyclodextrin compound is preferably selected from the group consisting of α-cyclodextrin, β-cyclodextrin, γ-cyclodextrin, hydroxypropyl-cyclodextrin, methyl-cyclodextrin, and glucosyl-cyclodextrin.

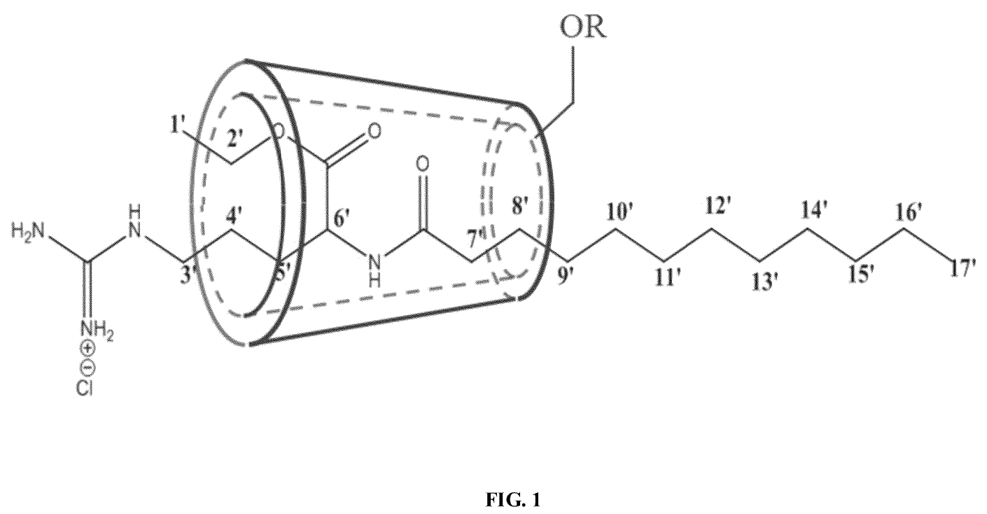

In the present disclosure, taking the LAE compound being the LAE hydrochloride as an example, a structural schematic diagram of the cyclodextrin-based LAE clathrate is shown in FIG. 1, where R is selected from the group consisting of —CH₂CH(OH)CH₃, —H, —CH₃, and —C₅H₁₂O₆. In the cyclodextrin-based LAE clathrate, the cyclodextrin compound is mainly clathrated in an ester group-acylamino segment of the LAE compound.

The present disclosure further provides a preparation method of the cyclodextrin-based LAE clathrate, including the following steps:

mixing the cyclodextrin compound, the LAE compound, and water, and conducting clathration to obtain the cyclodextrin-based LAE clathrate.

In the present disclosure, unless otherwise specified, all raw material components are commercially available products well known to persons skilled in the art.

In the present disclosure, the cyclodextrin compound and the LAE compound are at a molar ratio of preferably (0.5-6):1, more preferably (1-5):1, even more preferably (2-4):1, and the most preferably 3:1. The cyclodextrin compound and the LAE compound are the same as the aforementioned cyclodextrin compound and LAE compound, and will not be repeated here.

In the present disclosure, there is no special limitation on the mixing, as long as the cyclodextrin compound and the LAE compound can be dissolved in water, such as stirring. The mixing preferably specifically includes: dissolving the cyclodextrin compound in the water to obtain a cyclodextrin compound solution; and dissolving the LAE compound in the cyclodextrin compound solution to obtain a mixed solution. The cyclodextrin compound solution has a mass concentration of preferably 1% to 10%, more preferably 2% to 8%, and even more preferably 3% to 5%. In the mixed solution, the LAE compound has a mass concentration of preferably 0.5% to 5%, more preferably 1% to 4%, and even more preferably 1% to 3%.

In the present disclosure, the clathration is conducted at preferably 25° C. to 80° C., more preferably 30° C. to 70° C., and even more preferably 40° C. to 60° C. for preferably 5 min to 24 h. During the clathration, a hydrophobic part of the LAE compound enters a hydrophobic cavity of the cyclodextrin compound and occupies the hydrophobic cavity, such that water molecules with a high enthalpy in the hydrophobic cavity are released, thereby forming the clathrate.

In the mixed solution, the clathration is preferably conducted under stirring, high-speed dispersion, ultrasonic treatment, or high-pressure micro-jet. In the present disclosure, the stirring is conducted at preferably 300 rpm to 900 rpm, more preferably 400 rpm to 800 rpm, and even more preferably 500 rpm to 700 rpm for preferably 5 h to 24 h, more preferably 10 h to 20 h, and even more preferably 15 h to 20 h. The high-speed dispersion is conducted at preferably 10,000 to 18,000 rpm, more preferably 12,000 to 16,000 rpm, and even more preferably 14,000 to 15,000 rpm for preferably 5 min to 20 min, more preferably 8 min to 18 min, and even more preferably 10 min to 15 min. The ultrasonic treatment at preferably 200 W to 750 W, more preferably 300 W to 700 W, and even more preferably 400 W to 600 W for preferably 5 min to 10 min, more preferably 6 min to 9 min, and even more preferably 7 min to 8 min. The high-pressure micro-jet is conducted at preferably 60 MPa to 100 MPa, more preferably 70 MPa to 90 MPa, and even more preferably 80 MPa to 90 MPa for preferably 3 to 7 cycles, more preferably 4 to 6 cycles, and even more preferably 5 to 6 cycles.

In the present disclosure, the preparation method further includes preferably the following step after the clathration is completed: drying a reaction solution obtained from the clathration to obtain the cyclodextrin-based LAE clathrate. The drying includes preferably freeze-drying, spray-drying, or vacuum-drying. The freeze-drying is conducted at preferably −80° C. to −60° C., more preferably −80° C. to −70° C.; there is no special limitation on a freeze-drying time, as long as the reaction solution is dried to a constant weight. The spray-drying is conducted in an atomizer at a pressure of preferably 0.7 MPa to 1.25 MPa, more preferably 1 MPa to 1.2 MPa; the atomizer has an inlet temperature of preferably 140° C. to 180° C., more preferably 150° C. to 160° C., an outlet temperature of preferably 90° C. to 110° C. more preferably 100° C., and a rotational frequency of preferably 20 Hz to 40 Hz, more preferably 30 Hz; a feeding speed of the reaction solution is controlled by a rotating speed of a feed pump, and the rotating speed of the feed pump is preferably 15 rpm to 30 rpm, more preferably 20 rpm to 25 rpm; there is no special limitation on a spray-drying time, as long as the reaction solution is dried to a constant weight. The vacuum-drying includes preferably conducting refrigeration, alcohol washing, and vacuum-drying successively; the refrigeration is conducted at preferably −4° C. to 6° C., more preferably 0° C. to 4° C. for preferably 12 h to 48 h, more preferably 24 h to 30 h; an alcohol for the alcohol washing includes preferably one or more of ethanol, propylene glycol, and n-butanol; the alcohol washing is conducted preferably 1 to 8 times, more preferably 3 to 5 times; the vacuum-drying is conducted under a vacuum degree of preferably 0.05 MPa to 0.09 MPa, more preferably 0.06 MPa at preferably 50° C. to 90° C. more preferably 85° C.; there is no special limitation on a vacuum-drying time, as long as the reaction solution is dried to a constant weight.

The present disclosure further provides use of the cyclodextrin-based LAE clathrate or a cyclodextrin-based LAE clathrate prepared by the preparation method as an additive in food or cosmetics. The cyclodextrin-based LAE clathrate is added at preferably less than or equal to 0.02 wt %, more preferably 0.001 wt % to 0.02 wt %, and even more preferably 0.01 wt % to 0.015 wt % in the food. The cyclodextrin-based LAE clathrate is added at preferably 0.4 wt % to 0.8 wt %, more preferably 0.5 wt % to 0.7 wt %, and even more preferably 0.5 wt % to 0.6 wt % in the cosmetics. The cyclodextrin compound clathrated outside a middle section of the LAE compound forms a steric hindrance effect, which reduces the interaction between head-end cations of the LAE compound and the anion components. As a result, the antibacterial properties of the LAE compound are significantly improved. In addition, the cyclodextrin-based LAE clathrate combines the amphiphilic structure with a cationic hydrophilic head and a long-chain hydrophobic tail of the LAE compound, as well as the special structure of the cyclodextrin compound with a hydrophilic outer wall and a hydrophobic inner wall of the cavity. This significantly improves an emulsification performance and a low-temperature solubility of the LAE compound, and reduces a self-aggregation effect of the LAE compound. This can also significantly improve the thermal stability, acid-base stability, and low-temperature storage stability of the LAE compound, thereby expanding an application range of the LAE compound in food and cosmetics.

The technical solutions of the present disclosure will be clearly and completely described below with reference to the examples of the present disclosure. Apparently, the described examples are only a part of, not all of, the examples of the present disclosure. All other examples obtained by a person of ordinary skill in the art based on the examples of the present disclosure without creative efforts shall fall within the protection scope of the present disclosure.

### Example 1

At a room temperature, 3 g of a hydroxypropyl-β-cyclodextrin (HPβCD) powder was dissolved in 100 mL of pure water to obtain a hydroxypropyl-β-cyclodextrin solution (with a mass concentration of 3% w/v). The HPβCD solution was added to fully dissolve 1 g of an LAE hydrochloride (LAE·HCl) powder, and then subjected to clathration at 60° C. under magnetic stirring at 500 rpm for 5 h. An obtained reaction solution was spray-dried to a constant weight to obtain a cyclodextrin-based LAE clathrate (referred to as an HPβCD/LAE clathrate). The spray-drying was conducted in an atomizer at an inlet temperature of 150° C., an outlet temperature of 100° C.′, and a rotational frequency of 20 Hz to 40 Hz; a rotating speed of a feed pump was 25 rpm.

### Example 2

At a room temperature, 3 g of a γ-cyclodextrin (γ-CD) powder was dissolved in 100 mL of pure water to obtain a γ-cyclodextrin solution (with a mass concentration of 3% w/v). The γ-CD solution was added to fully dissolve 2 g of an LAE citrate powder, and then subjected to clathration at 25° C. under magnetic stirring at 700 rpm for 24 h. An obtained reaction solution was refrigerated at −4° C. for 24 h, washed with ethanol 4 times, and then vacuum-dried to a constant weight at 0.06 MPa and 85° C. to obtain a cyclodextrin-based LAE clathrate (referred to as a γ-CD/LAE clathrate).

### Example 3

At a room temperature, 6 g of an α-cyclodextrin (α-CD) powder was dissolved in 100 mL of pure water to obtain an α-cyclodextrin solution (with a mass concentration of 6% w/v). The α-CD solution was added to fully dissolve 0.5 g of LAE, subjected to high-pressure micro-jet at 25° C. and 100 MPa for 7 cycles, and then freeze-dried at −80° C. for 48 h to obtain a cyclodextrin-based LAE clathrate (referred to as an α-CD/LAE clathrate).

### Comparative Example 1

3 g of a hydroxypropyl-β-cyclodextrin powder was mixed with 1 g of an LAE hydrochloride powder evenly to obtain a physical mixture of the HPβCD/LAE.

### Test Example 1

### Structural Characterization

(1) Infrared spectroscopy: infrared spectrograms of the raw materials and the prepared product (the HPβCD, LAE, physical mixture of HPβCD/LAE, and HPβCD/LAE clathrate) in Example 1 and Comparative Example 1 were measured with a Fourier transform infrared spectrophotometer (Fourier transform infrared Spectroscopy, FT-IR). The test range was (400 to 4,000) cm⁻¹, a number of scans was 32, and a resolution was 4 cm⁻¹.

(2) The crystal structures of the raw materials and the prepared product samples in Example 1 and Comparative Example 1 were analyzed by an X-ray diffractometer (XRD). The crystal structures of the HPβCD, LAE, physical mixture of HPβCD/LAE, and HPβCD/LAE clathrate were determined within a diffraction angle of 20=5º to 40°. The test conditions included: a scanning speed of 2°/min, a tube pressure of 40 kV, and a tube current of 45 mA.

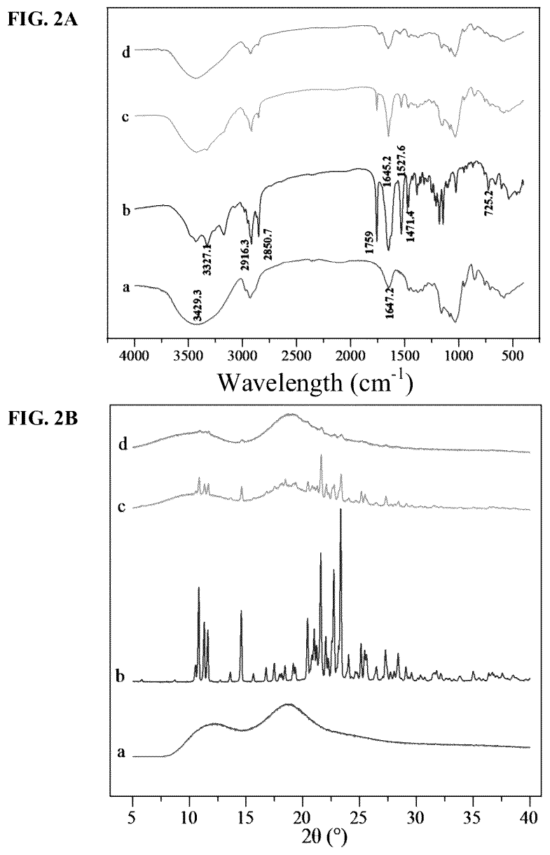

FIG. 2 showed Fourier transform infrared spectrograms (A) and XRD patterns (B) of raw materials (HPβCD and LAE) and prepared products in Example 1 and Comparative Example 1; where a was the HPβCD, b was the LAE, c was the physical mixture of the HPβCD/LAE, and d was the HPβCD/LAE clathrate. As shown in FIG. 2A, the physical mixture of the HPβCD/LAE was mainly characterized by superposition of the characteristic absorption peaks of these two substances. However, in the FT-IR spectrum of the HPβCD/LAE clathrate, the characteristic absorption peaks of the long carbon chain, amide, and ester groups of the LAE were significantly weakened or even almost disappeared. This was because the microenvironment changed, these groups entered the cavity of HPβCD, and the molecular vibration was restricted, such that the original infrared characteristics could not be fully displayed. In the clathrate spectrum, the —OH stretching vibration peak of HPβCD located at 3.429.3 cm⁻¹ was weakened and shifted to low frequency. This was because the hydroxyl group in HPβCD formed a hydrogen bond with a group of the LAE, such as a carbonyl group. At 1.647.2 cm⁻¹, the vibration peak of water molecules in the cavity of cyclodextrin weakened. This indicated that the decrease of water molecule content in the clathrate was due to the fact that a non-polar part of the LAE entered the cavity and squeezed the originally-bound polar water molecules out of the cavity of the cyclodextrin. The above results showed that the hydrophobic parts such as amide bonds, ester groups, and carbon chains in the LAE structure entered the HPβCD cavity with the release of high-energy water molecules in the cavity as a driving force, and combined with the hydrophobic cavity of HPβCD by means of hydrogen bonds and hydrophobic interactions to form a clathrate. It showed that the clathrate was successfully prepared by the present disclosure. As shown in FIG. 2B, the LAE exhibited many sharp characteristic diffraction peaks, the HPβCD exhibited broad amorphous diffraction peaks, and the physical mixture of the HPβCD LAE obviously exhibited a simple superposition of the characteristic peaks of LAE and HPβCD. In the diffraction pattern of the HPβCD LAE clathrate, the crystal diffraction peak of the LAE was weakened or even almost disappeared, indicating that the present disclosure successfully prepared the clathrate in an amorphous form.

(3) The ¹H NMR structure identification of the sample was conducted by an NMR spectrometer. 10 mg to 20 mg of the raw materials and the HPβCD/LAE clathrate sample in Example 1 were dissolved in a deuterated methanol solvent, and the ¹H NMR spectra of HPβCD. LAE, and HPβCD/LAE clathrate were measured, respectively. ¹H NMR can provide useful information for structural analysis of HPβCD and guest molecules in the clathrate. Whether a clathrate is formed can be determined by the changes of chemical shift of the clathrate before and after clathration. This is one of the most direct evidences to determine the clathrate structure. When the guest molecules enter the cavity of HPβCD, protons (H-3 and H-5) inside the cavity are relatively more sensitive to environmental changes than protons (H-1, H-2, and H-4) outside. Therefore, the H-3 and H-5 can be used as spectroscopic probes to study the presence of guest molecules and the interaction of host-guest molecules.

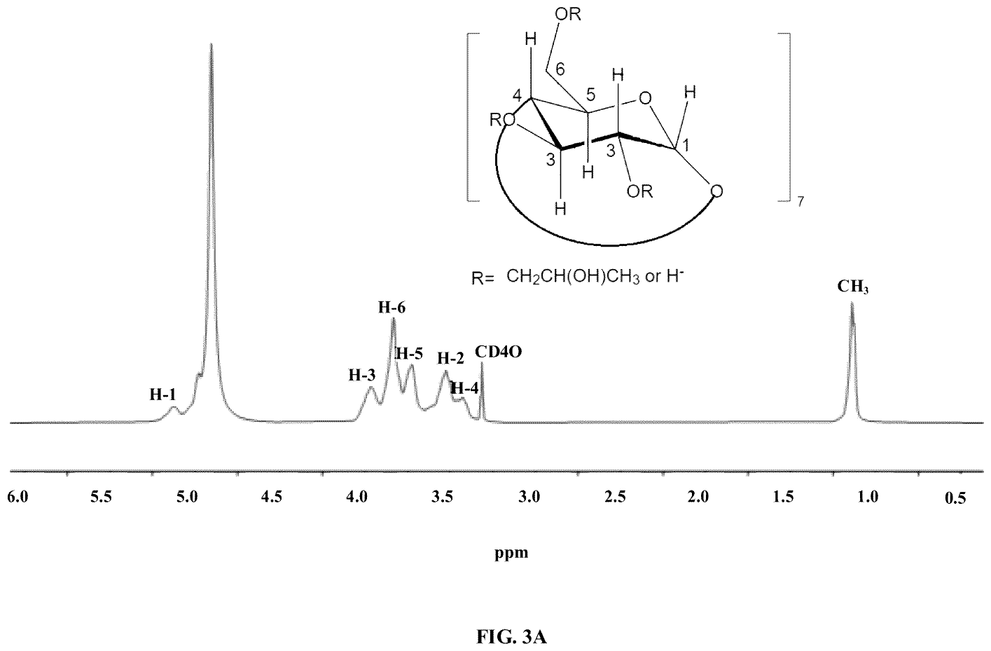

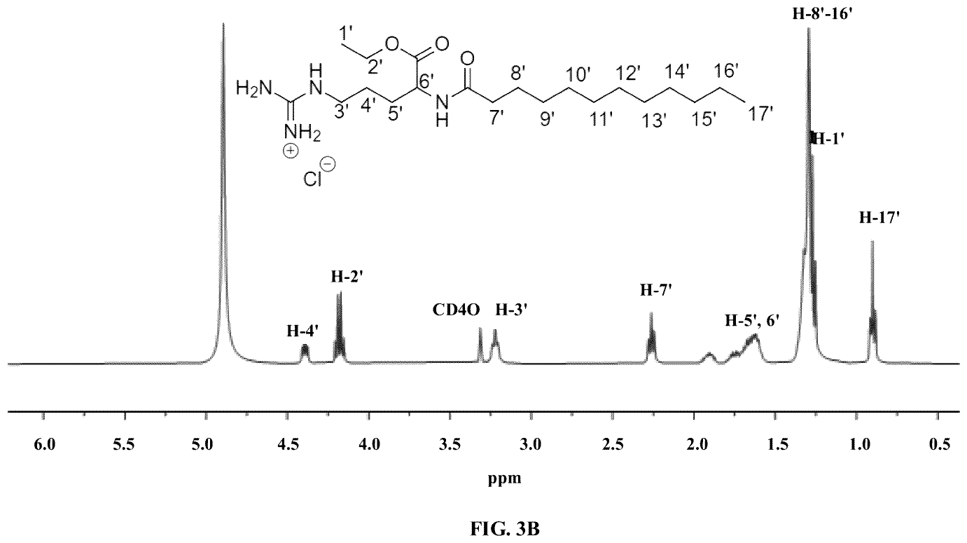

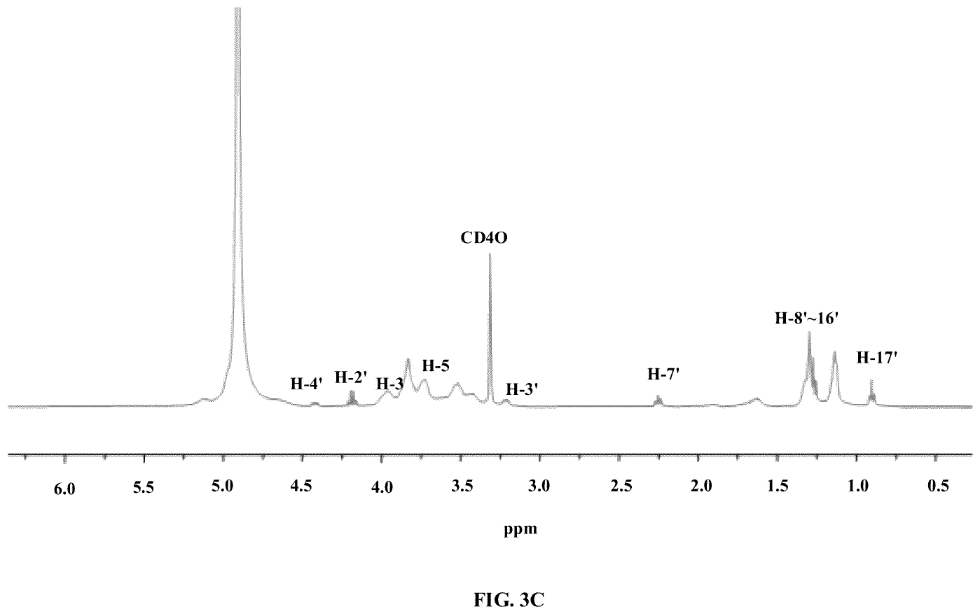

FIG. 3 showed 1H NMR spectrograms of the raw materials and the HPβCD/LAE clathrate in Example 1. As shown in FIG. 1, after HPβCD clathrated LAE, the relative changes in chemical shift of protons near the ester group and amide bond of LAE reached the maximum, indicating that the HPβCD mainly clathrated the LAE at the ester group and amide bond positions of LAE. After the clathrate was formed, H-3 and H-5 located in the cavity of HPβCD had relatively large chemical shifts, and the difference in chemical shifts of H-3 was greater than that of H-5. Since H-5 was located at the small-mouth end of the cavity, and H-3 was close to the large-mouth end of the cavity, it could be explained that the LAE penetrated into the cavity from the large-mouth end of HPβCD. The downfield shift of H-5 was caused by the hydrogen bond association, indicating that the LAE had penetrated deep into the cavity. The shift at H-6 might be due to the deflection of —CH₂ at the C-6 position of cyclodextrin after the clathration of LAE, which caused the long carbon chain of LAE penetrate out of the cavity of the HPβCD. In view of this, it was presumed that a structure of the clathrate was the structure schematically shown in FIG. 1.

### Test Example 2

### Antibacterial Properties

In order to test the performance of the HPβCD LAE clathrate prepared in Example 1, the antibacterial activity of the HPβCD/LAE clathrate in the presence of an anionic polysaccharide (xanthan gum) as an interfering substance was measured by an Oxford cup method. Groups A to G were set up, and each group had 3 parallel experiments. 15 mL of nutrient agar was added to each petri dish, cooled and solidified, and 100 μL of a Staphylococcus aureus solution (10⁵ CFU/mL) was spread on each petri dish. The B to G groups were further evenly coated with 0.5 mL of a 1% w/v xanthan gum solution (while the group A did not add the xanthan gum), and each group was added with 5 mL of agar to seal the cover. Each group was placed with an Oxford cup, and 130 μL of an antibacterial solution was added to each Oxford cup; each of the Oxford cups was diffused in a 4° C. refrigerator for 24 h, cultured at 37° C. for 24 h, and an inhibition zone diameter (IZD) was measured with a micrometer. The mass percentages of the antibacterial solution added in Groups A to G were as follows: A: 0.20% LAE-no xanthan gum; B: 0.20% LAE; C: 0.08% HPβCD/LAE clathrate-0.02% LAE; D: 0.16% HPβCD/LAE clathrate-0.04% LAE; E: 0.40% HPβCD/LAE clathrate-0.10% LAE; F: 0.60% HPβCD/LAE clathrate-0.15% LAE; and G: 0.80% HPβCD/LAE clathrate-0.20% LAE. The LAB was the raw material in Example 1.

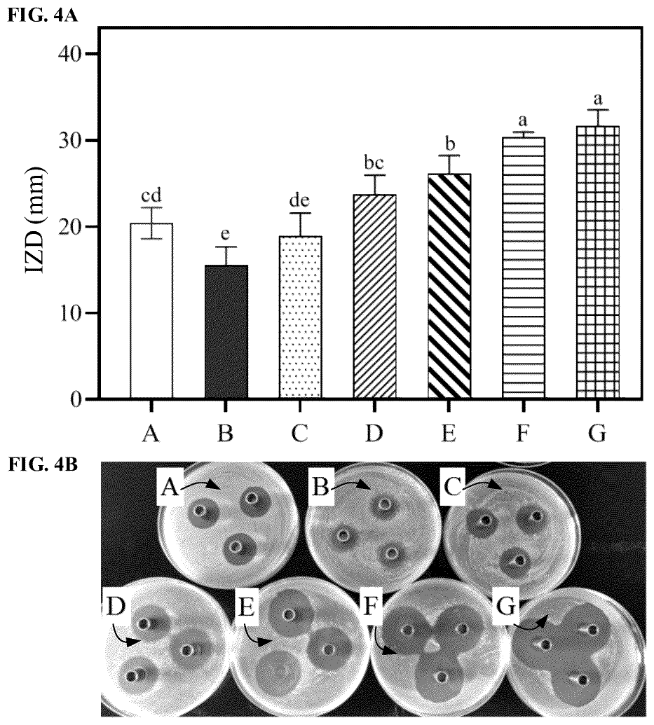

FIG. 4 showed an antibacterial activity of the HPβCD LAE clathrate to Staphylococcus aureus when xanthan gum was used as an interfering substance. It was seen from FIG. 4 that the xanthan gum significantly reduced the inhibition zone of LAE and weakened the antibacterial effect of LAE. The size of the inhibition zone of 0.08% HPβCD/LAE clathrate-0.02% LAE group was almost close to that of the 0.2% LAE-no xanthan gum group. As the concentration of HPβCD/LAE clathrate increased to 0.8%, the inhibition zone continued to increase and was larger than that of the LAE-no xanthan gum. This showed that the steric hindrance effect of HPβCD clathrate effectively reduced the reaction between LAE cations and macromolecular anion polysaccharides, and reduced the aggregation of LAE molecules themselves. In this way, the LAE interacted with the microorganisms more uniformly and fully, thereby significantly improving the antibacterial properties of the LAE.

### Test Example 3

### Emulsifying Properties and Stability

The emulsifying properties of the HPβCD/LAE clathrate prepared in Example 1 was evaluated by spectrophotometry, while the raw material LAE in Example 1, medium-chain triglyceride (MCT), and Tween-80 were used as controls. The LAE, MCT, Tween-80, and HPβCD/LAE clathrate were separately diluted 100 times with a 0.1% w/v sodium dodecyl sulfate solution to obtain emulsions to be tested. The absorbance (EA) of these emulsions to be tested was measured at 500 nm to evaluate the emulsifying ability.

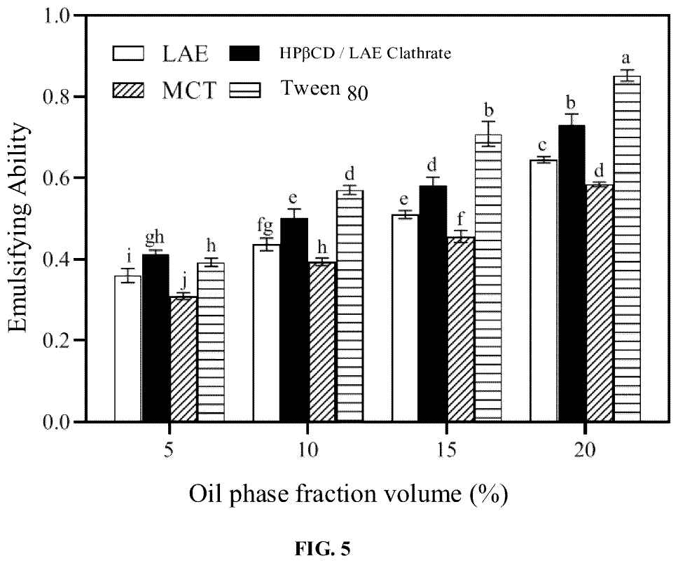

FIG. 5 showed a comparison result of emulsifying properties of the HPβCD LAE clathrate with other emulsifiers. As shown in FIG. 5, the emulsifying ability of HPβCD/LAE clathrate was significantly stronger than that of LAE; the emulsifying ability of HPβCD/LAE clathrate was stronger than that of MCT, and slightly weaker than that of the strong emulsifier Tween-80. This indicated that the HPβCD LAE clathrate of the present disclosure could be used as an emulsifier.

### Test Example 4

### Stability

The pH stability of a system was characterized by measuring turbidity and particle size. A 1% w/v LAE aqueous solution and an HPβCD/LAE clathrate aqueous solution (4% w/v) containing a same amount of 1% w/v LAE were prepared, respectively. The pH values of the LAE aqueous solution and the HPβCD/LAE clathrate aqueous solution were adjusted to 1, 3, 5, 7, 9, and 11 with 0.01 mol/L to 0.5 mol/L of sodium hydroxide and hydrochloric acid solutions, respectively. After allowing to stand at room temperature for 24 h, the absorbance at 600 nm of the LAE aqueous solutions and the HPβCD/LAE clathrate aqueous solutions with different pH values were measured with a UV spectrophotometer. The absorbance was used to represent a turbidity of the solution, while pure water was used as a reference. The particle size of the solution was measured with a ZS90 nanometer particle size analyzer. Photographs were taken to record the changes of the above solutions stored at 4° C. for 0 h, 24 h, 7 d, and 35 d to characterize the low-temperature storage stability of the samples.

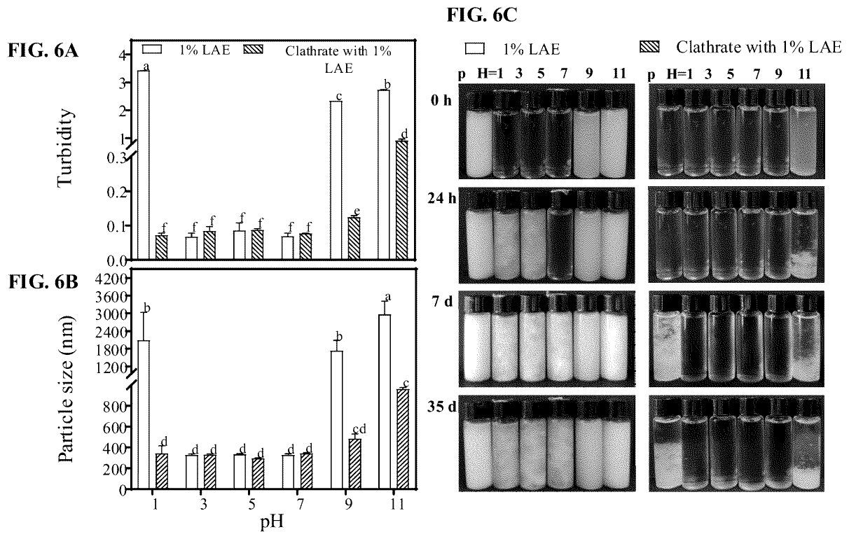

FIG. 6 shows a turbidity (A), a particle size (B), and a low-temperature storage appearance (C) of the raw material LAE and the HPβCD LAE clathrate in each of the examples at a pH value of 1 to 11. As shown in FIG. 6, the LAE obviously aggregated outside the pH value of 3 to 7, while the HPβCD/LAE clathrate was stable in the pH value of 1 to 9, showing a clear and transparent solution. This showed that the HPβCD/LAE clathrate of the present disclosure had an excellent pH stability. This was because the HPβCD with strong stability was clathrated outside the LAE, and the direct contact between LAE molecules was reduced due to steric hindrance. Thus, the aggregation was effectively reduced, such that the HPβCD/LAE clathrate had an excellent acid-base stability. After storage at low temperature for 24 h. except for the solution of pH=7, all other LAE solutions had obvious crystallization. With the prolongation of storage time at low temperature, LAE were eventually fully precipitated. This indicated that the low-temperature storage stability of the LAE solution was high. This was also the main limitation of the use of LAE in many types of food, such as refrigerated beverages, condiments, and desserts. However, after the HPβCD/LAE clathrate solution was stored at 4° C. for 35 d, the solution still remained transparent within the pH value of 3 to 9. This indicated that the HPβCD/LAE clathrate had an excellent storage stability at low temperature.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1. A cyclodextrin-based ethyl lauroyl arginate (LAE) clathrate, comprising a cyclodextrin compound and an LAE compound penetrating a cavity of the cyclodextrin compound.

2. The cyclodextrin-based LAE clathrate according to claim 1, wherein the LAE compound is selected from the group consisting of LAE and an LAE salt.

3. The cyclodextrin-based LAE clathrate according to claim 2, wherein the LAE salt is selected from the group consisting of LAE hydrochloride, LAE lactate, LAE citrate, LAE ascorbate, and an LAE fatty acid salt.

4. The cyclodextrin-based LAE clathrate according to claim 1, wherein the cyclodextrin compound is selected from the group consisting of α-cyclodextrin, β-cyclodextrin, γ-cyclodextrin, hydroxypropyl-cyclodextrin, methyl-cyclodextrin, and glucosyl-cyclodextrin.

5. A preparation method of the cyclodextrin-based LAE clathrate according to claim 1, comprising the following steps:
mixing the cyclodextrin compound, the LAE compound, and water, and conducting clathration to obtain the cyclodextrin-based LAE clathrate.

6. The preparation method according to claim 5, wherein the cyclodextrin compound and the LAE compound are at a molar ratio of (0.5-6):1.

7. The preparation method according to claim 5, wherein the mixing specifically comprises: dissolving the cyclodextrin compound in the water to obtain a cyclodextrin compound solution; and dissolving the LAE compound in the cyclodextrin compound solution.

8. The preparation method according to claim 7, wherein the cyclodextrin compound solution has a mass concentration of 1% to 10%.

9. The preparation method according to claim 5, wherein the clathration is conducted at 25° C. to 80° C. for 5 min to 24 h.

10. The preparation method according to claim 5, wherein the clathration is conducted under stirring, high-speed dispersion, ultrasonic treatment, or high-pressure micro-jet;
the stirring is conducted at 300 rpm to 900 rpm for 5 h to 24 h;
the high-speed dispersion is conducted at 10,000 rpm to 18,000 rpm for 5 min to 20 min;
the ultrasonic treatment is conducted at 200 W to 750 W for 5 min to 10 min; and
the high-pressure micro-jet is conducted at 60 MPa to 100 MPa for 3 to 7 cycles.

11. The preparation method according to claim 5, further comprising the following step after the clathration is completed: drying a reaction solution obtained from the clathration to obtain the cyclodextrin-based LAE clathrate.

12. The preparation method according to claim 11, wherein the drying comprises freeze-drying, spray-drying, or vacuum-drying;
the freeze-drying is conducted at −80° C. to −60° C.;
the spray-drying is conducted in an atomizer at a pressure of 0.7 MPa to 1.25 MPa; the atomizer has an inlet temperature of 140° C. to 180° C., an outlet temperature of 90° C. to 110° C., and a rotational frequency of 20 Hz to 40 Hz; a feeding speed of the reaction solution is controlled by a rotating speed of a feed pump, and the rotating speed of the feed pump is 15 rpm to 30 rpm; and
the vacuum-drying is conducted under a vacuum degree of 0.05 MPa to 0.09 MPa at 50° C. to 90° C.

13. A method for preparing food or cosmetics using cyclodextrin-based LAE clathrate according to claim 1 as an additive.

14. The use according to claim 13, wherein less than or equal to 0.02 wt % of the cyclodextrin-based LAE clathrate is added in the food; and
0.4 wt % to 0.8 wt % of the cyclodextrin-based LAE clathrate is added in the cosmetics.

15. The preparation method according to claim 5, wherein the LAE compound is selected form the group consisting of LAE and an LAE salt.

16. The preparation method according to claim 15, wherein the LAE salt is selected from the group consisting of LAE hydrochloride, LAE lactate, LAE citrate, LAE ascorbate, and an LAE fatty acid salt.

17. The preparation method according to claim 5, wherein the cyclodextrin compound is selected from the group consisting of α-cyclodextrin, β-cyclodextrin, γ-cyclodextrin, hydroxypropyl-cyclodextrin, methyl-cyclodextrin, and glucosyl-cyclodextrin.

18. The preparation method according to claim 9, wherein the clathration is conducted under stirring, high-speed dispersion, ultrasonic treatment, or high-pressure micro-jet;
the stirring is conducted at 300 rpm to 900 rpm for 5 h to 24 h;
the high-speed dispersion is conducted at 10,000 rpm to 18,000 rpm for 5 min to 20 min;
the ultrasonic treatment is conducted at 200 W to 750 W for 5 min to 10 min; and
the high-pressure micro-jet is conducted at 60 MPa to 100 MPa for 3 to 7 cycles.

19. The method according to claim 13, wherein the LAE compound is selected from the group consisting of LAE and an LAE salt.

20. The method according to claim 19, wherein the LAE salt is selected from the group consisting of LAE hydrochloride, LAE lactate, LAE citrate, LAE ascorbate, and an LAE fatty acid salt.
